(19) United States
(12) Patent Application Publication
(10) Pub. No.: US 2013/0224290 A1
(43) Pub. Date: Aug. 29, 2013

(54) Oral Pharmaceutical Composition of Duloxetine

(71) Applicants: Bandi Parthasaradhi Reddy, Hyderabad (IN); Podili Khadgapathi, Hyderabad (IN); Sanikommu Venkata Ramana Reddy, Hyderabad (IN); Kalidindi Praveen Kumar, Hyderabad (IN)

(72) Inventors: Bandi Parthasaradhi Reddy, Hyderabad (IN); Podili Khadgapathi, Hyderabad (IN); Sanikommu Venkata Ramana Reddy, Hyderabad (IN); Kalidindi Praveen Kumar, Hyderabad (IN)

(73) Assignee: HETERO RESEARCH FOUNDATION, Hyderabad (IN)

(21) Appl. No.: 13/699,699

(22) PCT Filed: May 25, 2010
(86) PCT No.: PCT/IN10/00353

§ 371 Date: May 10, 2013

(51) Int. Cl. A61K 9/16; A61K 31/381

(52) U.S. Cl. CPC A61K 9/1676; A61K 31/381
USPC 424/456; 424/400; 514/438; 424/494

(57) ABSTRACT

The present invention relates to an oral pharmaceutical composition of duloxetine or pharmaceutically acceptable salts thereof. Preferably, the invention relates to a delayed release composition of duloxetine comprising a core containing duloxetine, an optional separating layer, an enteric layer and an optional finishing layer.

### FIELD OF THE INVENTION

The present invention relates to an oral pharmaceutical composition of duloxetine or pharmaceutically acceptable salts thereof. The invention also relates to a delayed release composition of duloxetine comprising a core containing duloxetine, an optional separating layer, an enteric layer and an optional finishing layer.

### BACKGROUND OF THE INVENTION

Duloxetine is a selective serotonin and norepinephrine reuptake inhibitor (SSNRI), effective for major depressive disorder and it is as effective as venlafaxine in generalized anxiety disorder.

Duloxetine is chemically (+)-(S)-N-methyl-γ-(1-naphthyloxy)-2-thiophene propylamine, and commonly used as its hydrochloride salt. In this document, the term “duloxetine” will refer to the hydrochloride salt of the S-enantiomer unless otherwise specified. Duloxetine hydrochloride has the following formula.

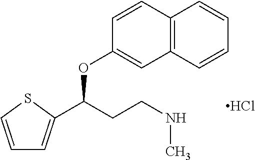 [SMILES: CNCC[C@H](Oc1ccc2ccccc2c1)c1cccs1 ; Cl]

Duloxetine is commercially available as capsules containing delayed release pellets under the trade name CYMBALTA™ in the United States. It has been approved by the FDA for the treatment of major depressive disorder, treatment of generalized anxiety disorder, management of neuropathic pain associated with diabetic peripheral neuropathy and management of fibromyalgia. Duloxetine is also commercially available as hard gastro-resistant capsules under the trade names CYMBALTA™ and YENTREVE™ in Europe. It has been approved by EMEA for the treatment of major depressive disorder, treatment of diabetic peripheral neuropathic pain in adults and treatment of generalized anxiety disorder under the brand name CYMBALTA™ and for the treatment of moderate to severe stress urinary incontinence in woman under the trade name YENTREVE™.

Duloxetine, being an acid-labile substance is very much susceptible to degradation in the acidic environment of the stomach. Therefore duloxetine is formulated as an enteric coated dosage form to protect it from acid degradation.

U.S. Pat. No. 5,508,276 discloses an enteric duloxetine pellet comprising hydroxypropylmethyl cellulose acetate succinate (HPMCAS) as an enteric coating polymer. The '276 patent also discloses that the HPMCAS should be neutralized, for example, with ammonia to facilitate its dissolution. The '276 patent also discloses that duloxetine was found to react with many enteric coatings to form a slowly soluble or insoluble coating. This may lead to a disadvantageous drug-releasing profile and/or low bioavailability.

US patent application no. 2006/0165776 describes an oral pharmaceutical composition comprising a core comprising duloxetine or its pharmaceutically acceptable derivative thereof and the said core comprised of pharmaceutically inert nuclei and duloxetine or its pharmaceutically acceptable derivative thereof mixed and compressed together, an intermediate layer and an enteric layer comprising one or more enteric polymers; wherein the said composition is free of alkaline reacting compounds.

US patent application no. 2007/0292511 discloses a duloxetine hydrochloride delayed release formulation, comprising an inert core, a drug layer comprising duloxetine hydrochloride, a separating layer and an enteric layer comprising at least one of methacrylic acid copolymer and hydroxypropyl methyl cellulose phthalate.

US patent application no. 2008/0226711 discloses a delayed release pharmaceutical composition comprising a core comprising an inert core coated with duloxetine, optionally a separating coat on the core and an enteric coat on the core or on the separating coat, wherein the enteric coat comprises hydroxypropyl methylcellulose phthalate (HPMCP) or cellulose acetate phthalate (CAP) or polyvinyl acetate phthalate (PVAP).

Jansen et al, J Pharm Sci, 87 (I), 1998: 81-85 discloses that duloxetine reacts with degradation products or residual free acids present in the enteric polymer such as hydroxypropyl methylcellulose phthalate to form impurities such as phthalamide impurities.

None of the above said prior art discloses the use of carboxymethyl ethyl cellulose as an enteric coating material in the delayed release pharmaceutical composition of duloxetine. The present invention overcomes the above commonly faced stability problems with the enteric coated formulations of duloxetine.

Accordingly, the present invention provides a delayed release composition comprising; inert core, a drug layer comprising duloxetine, an optional separating layer, an enteric layer comprising carboxymethyl ethyl cellulose and an optional finishing layer.

### OBJECTIVE OF THE INVENTION

Accordingly, the main objective of the invention is to provide a delayed release composition of duloxetine or its pharmaceutically acceptable salts comprising; inert core, a drug layer comprising duloxetine, an optional separating layer, an enteric layer comprising carboxy methyl ethyl cellulose and an optional finishing layer.

### SUMMARY OF THE INVENTION

Accordingly, the main aspect of the present invention is to provide a delayed release formulation comprising:
a) an inert core loaded with duloxetine or its pharmaceutically acceptable salts.
b) an optional separating layer.
c) an enteric coating over the sub coating with carboxymethyl ethyl cellulose; and
d) an optional finishing layer.

### DETAILED DESCRIPTION OF THE INVENTION

According to the present invention there is provided a delayed release pharmaceutical composition of duloxetine or its pharmaceutically acceptable salts comprising:
a) an inert core loaded with duloxetine or its pharmaceutically acceptable salts.
b) an optional separating layer.
c) an enteric coating over the sub coating with carboxy methyl ethyl cellulose; and
d) an optional finishing layer.

Preferably, duloxetine is in the form of its hydrochloride salt.

Preferably, the inert core comprises sugar spheres or pellets of microcrystalline cellulose and more preferably, the inert core comprises sugar spheres.

The drug layer further comprises one or more pharmaceutically acceptable excipients that do not react adversely with duloxetine.

Preferably, the pharmaceutically acceptable excipients are selected from diluents, binders and disintegrants.

The preferable diluent is selected from the group consisting of mannitol, sucrose, sorbitol, starch, modified starches, xylitol, lactose, microcrystalline cellulose, magnesium carbonate, starch, calcium carbonate, dicalcium phosphate, tribasic calcium phosphate, and calcium sulphate.

The preferable binder is selected from L-hydroxy propyl cellulose, corn starch, polyvinyl pyrrolidine, hydroxyl propyl methyl cellulose, hydroxyl ethyl cellulose and pre-gelatinized starch.

The disintegrant may be preferably selected from croscarmellose sodium, crospovidone, sodium starch glycolate and low substituted hydroxyl propyl cellulose.

More preferably, the pharmaceutically acceptable excipients are selected from sucrose, hydroxy propyl cellulose, hydroxy propyl methyl cellulose, crospovidone, talc and mixtures thereof.

Preferably, the drug layer comprises duloxetine, sugar spheres, hydroxy propyl methyl cellulose, hydroxy propyl cellulose, sucrose extra fine, crospovidone and talc.

More preferably, the drug layer comprises about 15% to about 40% duloxetine, about 30-80% sugar spheres, about 2-10% hydroxy propyl methyl cellulose, about 0-10% hydroxy propyl cellulose, about 0-10% sucrose extra fine, about 1-10% crospovidone and about 1-10% talc, wherein the percentages are by weight of the drug layer.

The separating layer between drug layer and enteric layer is optional. The functions of the separating layer, if applied, are to provide a smooth base for the application of the enteric layer, to prolong the pellet's resistance to acid conditions and to improve stability.

The preferable separating layer comprises sucrose, hydroxy propyl methyl cellulose and talc.

Preferably, the separating layer is present in an amount of about 5-30% based on the total weight of the formulation.

More preferably, the separating layer is present in an amount of about 10-25% based on the total weight of the formulation.

The enteric layer may preferably comprise carboxymethyl ethyl cellulose and povidone.

The enteric layer is present in an amount of about 5-30% based on the total weight of the formulation.

More preferably, the enteric layer is present in an amount of about 10-20% based on the total weight of the formulation.

The solvent used to make the enteric coating solution is selected from isopropanol, water and mixtures thereof. The preferable solvent is a mixture of isopropanol and water.

The preferable ratio of isopropanol and water used in the enteric coating solution is 1:9 to 9:1.

Optionally, a finishing layer is present over the enteric coating layer.

Preferably, the optional finishing layer comprises hydroxypropyl methyl cellulose, talc, polyethylene glycol 400 (PEG-400) and titanium dioxide.

Preferably, the delayed release pharmaceutical composition of duloxetine is in the form of pellets.

The film coated duloxetine pellets were then filled into hard gelatin capsules.

The following examples further exemplify the invention and are not intended to limit the scope of the invention.

### EXAMPLE 1

| Ingredient | Quantity (mg) |
|---|---|
| Core | |
| Sugar spheres | 155.70 |
| Duloxetine hydrochloride | 67.30 |
| Hydroxypropyl methyl cellulose | 10.00 |
| Hydroxy propyl cellulose | 3.00 |
| Sucrose extra fine | 3.00 |
| Crospovidone | 5.00 |
| Talc | 6.00 |
| Purified water | q.s |
| Separating layer | |
| Sucrose | 31.00 |
| Hydroxypropyl methyl cellulose | 11.00 |
| Talc | 11.00 |
| Purified water | q.s |
| Enteric Coating | |
| Carboxy methyl ethyl cellulose | 39.00 |
| Povidone | 3.00 |
| Isopropanol/Water (7:3) | q.s |
| Total weight | 345.00 |

### EXAMPLE 2

| Ingredient | Quantity (mg) |
|---|---|
| Core | |
| Sugar spheres | 155.70 |
| Duloxetine hydrochloride | 67.30 |
| Hydroxypropyl methyl cellulose | 10.00 |
| Sucrose extra fine | 3.00 |
| Crospovidone | 5.00 |
| Talc | 6.00 |
| Purified water | q.s |
| Separating layer | |
| Sucrose | 31.00 |
| Hydroxypropyl methyl cellulose | 11.00 |
| Talc | 11.00 |
| Purified water | q.s |
| Enteric Coating | |
| Carboxy methyl ethyl cellulose | 39.00 |
| Povidone | 3.00 |
| Isopropanol/Water (8:2) | q.s |
| Total weight | 342.00 |

### EXAMPLE 3

| Ingredient | Quantity (mg) |
|---|---|
| Core | |
| Sugar spheres | 155.70 |
| Duloxetine hydrochloride | 67.30 |
| Hydroxypropyl methyl cellulose | 10.00 |
| Sucrose extra fine | 3.00 |
| Crospovidone | 5.00 |
| Talc | 6.00 |
| Purified water | q.s |
| Separating layer | |
| Sucrose | 22.00 |
| Hydroxypropyl methyl cellulose | 5.00 |
| Talc | 10.00 |
| Purified water | q.s |
| Enteric Coating | |
| Carboxy methyl ethyl cellulose | 41.00 |
| Povidone | 3.10 |
| Isopropanol/Water (7:3) | q.s |
| Total weight | 328.10 |

### EXAMPLE 4

| Ingredient | Quantity (mg) |
|---|---|
| Core | |
| Sugar spheres | 155.70 |
| Duloxetine hydrochloride | 67.30 |
| Hydroxypropyl methyl cellulose | 13.00 |
| Hydroxypropyl cellulose | 3.00 |
| Crospovidone | 5.00 |
| Talc | 6.00 |
| Purified water | q.s |
| Separating layer | |
| Sucrose | 42.40 |
| Hydroxypropyl methyl cellulose | 6.60 |
| Talc | 13.00 |
| Purified water | q.s |
| Enteric Coating | |
| Carboxy methyl ethyl cellulose | 41.80 |
| Povidone | 3.20 |
| Isopropanol/Water (7:3) | q.s |
| Total weight | 357.00 |

### EXAMPLE 5

| Ingredient | Quantity (mg) |
|---|---|
| Core | |
| Sugar spheres | 155.70 |
| Duloxetine hydrochloride | 67.30 |
| Hydroxypropyl methyl cellulose | 10.00 |
| Sucrose extra fine | 3.00 |
| Crospovidone | 5.00 |
| Talc | 6.00 |
| Purified water | q.s |
| Separating layer | |
| Sucrose | 32.00 |
| Hydroxypropyl methyl cellulose | 5.00 |
| Talc | 10.00 |
| Purified water | q.s |
| Enteric Coating | |
| Carboxy methyl ethyl cellulose | 41.00 |
| Povidone | 3.10 |
| Isopropanol/Water (7:3) | q.s |
| Total weight | 338.10 |

### Preparation Process

The process for preparation of formulations of examples 1-5 as given below:

### Core

Duloxetine hydrochloride and other inactive ingredients were dispersed in purified water. This dispersion was coated on the sugar spheres in a fluid bed processor.

### Separating Layer

Sucrose and hydroxypropyl methyl cellulose were dissolved in purified water and talc dispersed in to this solution under stirring. This dispersion was coated on the drug loaded spheres in a fluid bed processor.

### Enteric Layer

In order to provide enteric coated duloxetine pellets, the above coated particles were then coated in fluid bed processor with a solution of carboxymethyl cellulose, povidone in purified water and isopropanol.

### Finishing Layer

Hydroxypropyl methyl cellulose, polyethylene glycol, titanium dioxide and talc were added in purified water and this dispersion was coated on the enteric coated pellets in a fluid bed processor.

### Capsule Filling

The above coated pellets were then filled into hard gelatin capsules.

1. A delayed release pharmaceutical composition of duloxetine or its pharmaceutically acceptable salts comprising:
a) an inert core having disposed thereon a drug layer comprising duloxetine or its pharmaceutically acceptable salts.
b) an optional separating layer disposed on the inert core.
c) an enteric coating comprising carboxymethyl ethyl cellulose disposed on the inert core or the separating layer; and
d) an optional finishing layer disposed on the enteric coating.

2. The delayed release pharmaceutical composition of claim 1, wherein the duloxetine is in the form of its hydrochloride salt.

3. The delayed release pharmaceutical composition of claim 1, wherein the inert core comprises sugar spheres or pellets of microcrystalline cellulose.

4. (canceled)

5. The delayed release pharmaceutical composition of claim 1, wherein the drug layer comprises one or more pharmaceutically acceptable excipients that do not react adversely with duloxetine.

6. The delayed release pharmaceutical composition of claim 5, wherein the pharmaceutically acceptable excipient is selected from diluents, binders and disintegrants.

7. The delayed release pharmaceutical composition of claim 6, wherein the pharmaceutically acceptable diluent is selected from mannitol, sorbitol, sucrose, starch, modified starches, xylitol, lactose, microcrystalline cellulose, magnesium carbonate, starch, calcium carbonate, dicalcium phosphate, tribasic calcium phosphate, and calcium sulphate.

8-9. (canceled)

10. The delayed release pharmaceutical composition of claim 1, wherein the inert core comprises sugar spheres, and wherein the drug layer comprises duloxetine hydrochloride, hydroxypropyl methyl cellulose, hydroxypropyl cellulose, sucrose extra fine, crospovidone and talc.

11. The delayed release pharmaceutical composition of claim 1, wherein the coated inert core comprises 15% to about 40% duloxetine hydrochloride, about 30-80% sugar spheres, about 2-10% hydroxypropyl methyl cellulose, about 0-10% hydroxypropyl cellulose, about 0-10% sucrose extra fine, about 1-10% crospovidone and about 1-10% talc based on the weight of the drug layer.

12. The delayed release pharmaceutical composition of claim 1, wherein the composition comprises the separating layer and the separating layer comprises sucrose, hydroxypropyl methyl cellulose and talc.

13. The delayed release pharmaceutical composition of claim 1, wherein the composition comprises the separating layer and the separating layer is present in an amount of about 5-30% based on the total weight of the Formulation, and wherein the enteric layer is present in an amount of about 5-30% based on the total weight of the formulation.

14. (canceled)

15. The delayed release pharmaceutical composition of claim 1, wherein the enteric layer further comprises povidone.

16-18. (canceled)

19. The delayed release pharmaceutical composition of claim 18, wherein the composition comprises the finishing layer and the finishing layer comprises hydroxypropyl methyl cellulose, polyethylene glycol, titanium dioxide and talc.

20. (canceled)

21. The delayed release pharmaceutical composition of claim 1, wherein the delayed release pharmaceutical composition of duloxetine is in the form of pellets.

22. The delayed release pharmaceutical composition of claim 21, wherein the pellets were filled in hard gelatin capsules.
